(19) United States
(12) Patent Application Publication
(10) Pub. No.: US 2024/0194347 A1
(43) Pub. Date: Jun. 13, 2024

(54) Private AI Training

(71) Applicant: BIOTRONIK SE & Co. KG, Berlin (DE)

(72) Inventors: Thomas DOERR, Berlin (DE); Jens MUELLER, Berlin (DE); Matthias GRATZ, Erlangen (DE); R. Hollis WHITTINGTON, Portland, OR (US)

(73) Assignee: BIOTRONIK SE & Co. KG, Berlin (DE)

(21) Appl. No.: 18/553,579

(22) PCT Filed: Mar. 17, 2022
(86) PCT No.: PCT/EP2022/056961

§ 371 Date: Oct. 2, 2023

Related U.S. Application Data
(60) Provisional application No. 63/172,113, filed on Apr. 8, 2021.

(30) Foreign Application Priority Data
May 3, 2021 (EP) 21171771.5

(51) Int. Cl. G16H 50/20; G06F 8/30

(52) U.S. Cl. CPC G16H 50/20; G06F 8/30

(57) ABSTRACT

An artificial-intelligence based patient assessment system is provided for assessing a patient. The system comprises at least one AI based algorithm and means for providing computer code to a local data system, wherein the computer code is adapted to, when executed, process data to provide a training result for the at least one AI based algorithm, wherein the computer code is adapted to be executed in the local data system to process data stored in the local data system. The system further comprises means for receiving the training result from the local data system upon execution of the computer code in the local data system.

### CROSS-REFERENCE TO RELATED APPLICATIONS

This application is the United States National Phase under 35 U.S.C. § 371 of PCT International Patent Application No. PCT/EP2022/056961, filed on Mar. 17, 2022, which claims the benefit of European Patent Application No. 21171771.5, filed on May 3, 2021, and U.S. Provisional Patent Application No. 63/172,113, filed on Apr. 8, 2021, the disclosures of which are hereby incorporated by reference herein in their entireties.

### TECHNICAL FIELD

The present disclosure generally relates to systems and methods for assessing patients, in particular to training artificial intelligence (AI) based algorithms based on private patient data.

### BACKGROUND

It has been known to use centralized, server- or cloud-based medical data processing systems that assess patients based on data. For example, data collected by medical devices implanted into patients may be provided to such systems that process this data and determine a condition of the patient. Based on the medical-device-specific data provided from the respective medical devices, the patient can, for example, be continuously monitored or, more generally, a condition, such as a medical condition, of the patient can be determined. In particular, unexpected or otherwise unnoticed events may thus be detected.

It would be desirable to implement artificial intelligence (AI) based algorithms to a larger extent into such or similar systems. However, AI based algorithms tend to require large data sets for training. Due to strict privacy regulations, such systems often are not allowed to access patient data. Such patient data are typically stored in local data systems that may be installed or operated locally, such that also access may be provided locally only (such as within a hospital). Therefore, the training of known systems is typically limited to be based on data available to the systems, whereas locally stored and confidential patient data cannot be used to a larger extent. Alternatively, to use patient data, known systems sometimes are enabled to train based on sensitive, protected data in local studies (e.g., limited to local data systems) sometimes also based on separate contractual agreements.

Hence, the amount of accessible data sets is limited and/or the administrative efforts to make use of (local) data sets is enormous. There is therefore a need to improve patient assessment by the known systems.

This need is at least in part met by certain aspects of the present disclosure.

The present disclosure is directed toward overcoming one or more of the above-mentioned problems, though not necessarily limited to embodiments that do.

### SUMMARY

According to a first aspect, an artificial intelligence (AI) based patient assessment system for assessing a patient is provided. The system may comprise at least one AI based algorithm. The system may further comprise means for providing computer code to a local data system (e.g., a local patient data system). The computer code may be adapted to, when executed, process data (e.g., patient data) to provide a training result for the at least one AI based algorithm, wherein the computer code may be adapted to be executed in the local data system to process data stored in the local data system. The system may further comprise means for receiving the training result from the local data system upon execution of the computer code in the local data system.

The algorithm may, for example, be adapted to determine an (automatic) assessment (e.g., a certain diagnosis, a certain (medical) condition, a recommended action, and/or recommended decision) of a specific patient, e.g., based on input data associated with the patient. For example, the algorithm may be based on an artificial neural network. The input data may be any data that characterizes the (state of the) patient, e.g., it may include data characterizing the health state of the patient (e.g., blood pressure, heart rate) and/or indirect parameters associated therewith (e.g., humidity or temperature of the patient's environment), or it may relate to general patient data (e.g., date of birth, blood type). It may generally be based on static and/or dynamic patient data. The data may be retrieved from one or more devices associated with the patient (e.g., medical devices, such as implants etc., and/or a smartphone etc.), i.e. it may be device-specific and/or medical-device-specific. For example, the patient assessment system may (e.g., periodically, repeatedly or essentially in real-time) receive data obtained by a medical device (e.g., a medical device implanted into a patient, such as a cardiologic implant, e.g., a cardiac monitor, a pacemaker, a cardioverter-defibrillator (ICD) or a system for cardiac resynchronization therapy (CRT); a medical device attached to the patient, carried by the patient) or any other device (e.g., a smartwatch or any other wearable), e.g., carried by the patient. Additionally, the algorithm may also be based on input data retrieved from patient files, (medical and/or patient) data input by the patient themselves, etc. Based thereon, the algorithm may determine an assessment of the patient.

The patient assessment system may be server- and/or cloud-based. It may be implemented as a central processing system that may be globally accessible and that may provide patient assessment to patients and/or medical staff.

Hence, the data (which may be confidential or otherwise protected patient data) does not need to be exported from the local data system or otherwise leave the local data system (which may be a confidential or otherwise protected system and/or environment). Nevertheless, it may be used for training the AI based algorithm(s) of the patient assessment system and thus enhance the assessment of individual patients. Hence, a (globally accessible) patient assessment system may harness (only locally accessible) data for training, without the data having to leave its protected environment. Patient assessment is thus improved without compromising data privacy. Thus, a much wider range of training data may be used, potentially providing secure access to hundreds or thousands of local (hospital) data networks and, e.g., electronic health files stored therein, which associate input data with decisions/diagnoses/conditions/actions determined and/or recommended by highly trained physicians and/or other medical staff. This greatly enhances the functionality of the algorithm and its precision in predicting certain patient assessments. In particular, this allows to train basically in real-time such that the algorithms may be trained on the most recent data available.

For example, the patient assessment system may determine that the quality of a certain assessment may be improved (or an assessment may be enabled in the first place), if algorithms of the patient assessment system are further trained concerning one or more selected aspects. For example, the patient assessment system may determine that a certain assessment (e.g., a certain diagnosis, a certain (medical) condition, a recommended action, and/or recommended decision) of a specific patient may be likely, but the likelihood that the assessment Is correct may be improved based on certain aspects. The patient assessment system may then provide corresponding computer code to the local data system to process data locally within the local data system and to return a training result concerning the one or more selected aspects. The computer code may be patient-specific and/or specific to the respective aspects to be trained by the computer code. The data of the local data system may be processed by the computer code in a specific manner as needed, and a corresponding training result may be provided to the patient assessment system such that the latter may use it for assessing the patient. The training result may include, for example, one or more values for one or more predetermined weight factors to be applied in an artificial neural network.

The patient assessment system may of course not only assess a patient at a single point in time but also assess or monitor the patient periodically or otherwise repeatedly (e.g., within every second, every minute, every hour, every day; or according to a certain trigger event) or essentially in real-time. The computer code provided to the local data system may vary, for example, depending on the current algorithms implemented in the patient assessment system (these may be adapted to be self-learning and thus change) and/or depending on current data that can directly be accessed by the patient-assessment system (such as medical-device-specific data).

The computer code may be (directly) executable, such as in the form of a native code and/or a native application. It may also generally use principles of edge computing and/or edge applications and/or smart applications. The computer code may be provided with access rights to access (confidential) data stored in the local data system (or confidential parts thereof), if executed in the local data system. The functions of the patient assessment system may be implemented in an embedded application, for example. The embedded application may be based on AI algorithms, such as an artificial neural network, for example.

Deep learning may be used to train the neural network. Preferably, the neural network is a feed-forward network with multiple hidden layers or a recurrent neural network with multiple hidden layers. If a diagnostic therapeutic procedure requires, for instance, processing of image data, video data, or audio data, it is preferable to use a neural network with more than 2 dimensions (e.g., a convolutional neural network with 3 dimensions).

The AI algorithms optionally include input signal conditioning (filtering, scaling, normalization, transformation, etc.) and/or result post-processing (clustering, weighting, filtering, plausibility checking, etc.).

Optionally, the neural network may have a model control layer to make the medical application traceable (verification and monitoring of the model). The type of data used to train the neural network may depend largely on the desired medical support function.

The data of the local data system may include one or more patient data sets. For example, they may be stored in an electronic health file, e.g., in a hospital system. The patient data may only be locally accessed (e.g., within the local data system, e.g., disparate from the patient assessment system).

It is noted that the local data system needs not necessarily be a medical data system. Also applying the principles outlined herein to other local data systems may be conceivable that store (confidential) data associated with the patient to be assessed and/or other patients. Generally, assessing a patient may be based on any data that characterizes the patient, e.g., as outlined above. The local data system may be any local IT system, and/or IT environment, that may be secured and/or protected.

The patient assessment system may comprise one or more interfaces to export computer code to one or more local data systems and/or to receive training results from one or more local data systems. An interface may generally refer to a hardware and/or a software interface. A hardware interface may be understood as, e.g., a USB connection, an ethernet connection or any other suitable hardware interface which allows a transfer of computer code. The hardware interface may be based on a wireless (e.g., Wi-Fi, 5G, LTE, Bluetooth or any other suitable standard and/or air interface) and/or a wired connection (e.g., a copper wired connection), e.g., via the internet. Additionally or alternatively, the interface may also be a software interface, e.g., that allows exporting the computer code.

The local data system may comprise one or more similar interfaces to receive computer code from one or more patient assessment systems and/or to provide training results to one or more patient assessment systems.

In some examples, the patient assessment system further comprises means for (automatically) generating the computer code. The generating may be based on device-specific, e.g., medical-device-specific, and/or patient-specific data associated with the patient. Processing the data may reveal that further training concerning one or more selected aspects is needed to improve the present assessment based on the data. The code may then be generated adapted to train the selected aspect(s). For example, in order to obtain training data that may be helpful in order to further improve the result of the assessment based on heartbeat frequency values and/or ranges, it may be helpful to obtain further training data linking the (available) age, weight, and/or general fitness state of the patient to a certain diagnosis.

For example, the device-specific data may be transmitted from the device (possibly via an intermediate local transmitter) to a server of the patient assessment system and/or stored, there. The device-specific data may be globally accessible.

Additionally or alternatively, the patient assessment system may comprise means for generating the computer code adapted to a specific local data system (e.g., adapted to hardware and/or software and/or a data type used by the local system). It is also possible that the computer code is generated based on an access level that may be associated with one or more local data systems. For example, the local system(s) may be arranged as (parts of) individual hospitals and/or groups of hospitals. The computer code may then be adapted based on the specific (parts of) individual hospitals and/or groups of hospitals whose data the computer code is supposed to access. Similarly, the computer code may be generated based on a country, in which the local data system is to be accessed, e.g., based on data privacy regulations in the specific country. Hence, national and/or regional regulations may be taken into account.

Additionally or alternatively, it is also possible that the means for generating is adapted to generate the computer code, e.g., if a new local data system becomes available.

The patient assessment system may further comprise means for updating the at least one algorithm based on the training result. For example, if the algorithm is based on an artificial neural network, one or more weight factors of the network may be updated based on the training result. For example, the training result may comprise the updated one or more weight factors. In other examples, the patient assessment system may process one or more weight factors comprised by the training result in order to determine updated weight factors. For example, a weighted sum of one or more existing weight factors and one or more weight factors in the training result may be calculated to obtain one or more updated weight factors.

Further, the (updated) algorithm may be adapted for determining a (medical) condition of the patient, and/or a diagnosis and/or a recommended action and/or recommended medical decision for the patient, at least in part based on the training result. Specifically, the (updated) algorithm may operate on data available for a patient to assess the patient, e.g., on device-specific data and/or any data as described herein. For example, (updated) weight factors of a neural network may be applied to the data.

The patient assessment system may further comprise means for forwarding the determined medical condition (and/or the diagnosis, and/or the decision and/or the action) to the patient and/or to medical staff. The patient and/or medical staff may thus be informed about an unexpected condition and/or they may be kept up to date concerning the patient, e.g., in real-time. The patient and/or the medical staff may then react accordingly, without the patient having to proactively consult with the medical staff, first. Hence, unexpected and/or unnoticed events may be reacted to more quickly and efficiently.

For example, for a patient having an implanted cardiologic device and/or carrying a cardiologic device, the patient assessment system may diagnose a certain cardiologic condition and/or recommend a specific action.

The patient assessment system may comprise an interface to the local data system to trigger execution of the computer code provided to the local data system. For example, a trigger command may be sent in the same manner as the computer code, e.g., possibly at the same time or later.

In some examples, the algorithm and/or the updated algorithm may be adapted to attempt determining the medical condition of the patient (and/or the diagnosis and/or the recommended decision and/or the recommended action for the patient) based on medical-device-specific data or other available data associated with the patient. For example, medical-device-specific data available within the patient assessment system may be processed to this end. The means for generating may be adapted to generate, in case the medical condition of the patient (or the diagnosis/decision/action) cannot be determined (with a desired level of accuracy and/or certainty) based on the data associated with the patient, the computer code based on the data associated with the patient, wherein the computer code is adapted for obtaining training data required for determining the medical condition. For example, it may be determined that a condition/decision/action can only be determined with a limited level of accuracy and/or certainty or possibly not at all. In such case, it may be concluded that the determination may be improved (or enabled in the first place) by further training, e.g., of certain aspects, e.g., refining certain weight factors of a neural network.

For example, the patient assessment system may determine, based on the (device-specific) data associated with the patient, that further training, e.g., concerning a specific aspect, may be required. It may then generate the computer code adapted to process patient data to provide corresponding training, e.g., concerning the specific aspect. For example, it may generate computer code to provide a training result corresponding to weight factor(s) of a neural network corresponding to the aspect.

The patient assessment system may determine, based on the available (device-specific) data associated with the patient, that a determination of a medical condition of the patient (or a diagnosis/recommended decision/action for the patient) may be improved or enabled by looking at a certain aspect of patient data specific to the patient, e.g., for which no reasonable and/or only rough/inaccurate weight factor(s) have been determined. It may then generate computer code to provide a training result corresponding to these factor(s).

The means for providing the computer code may further be adapted to provide the computer code to the local data system in a secured and/or confidential manner. For example, a secure connection may be established. Also, the computer code may be secured using a Blockchain and/or suitable encryption scheme. Additionally or alternatively, the means for receiving the training result from the local data system may be adapted to the receive the training result in a secured and/or confidential manner. Also here, a secure connection (e.g., the same secure connection) and/or a Blockchain (e.g., the same Blockchain) may be used and/or suitable encryption scheme.

The patient assessment system may be adapted as a cardiologic monitoring and/or decision system. The patient assessment system may, for example, be adapted to receive data obtained by a cardiologic device, e.g., a cardiologic implant, e.g., as outlined herein, and/or to provide cardiologic assessments, e.g., as described herein.

The patient assessment system may comprise an (automatic) audit checking path, which may enable checks concerning the adherence to data privacy regulations (e.g., at any time).

Automatic cyclic checks include:
After a connection between 2 communication partners has been established, each incoming and outgoing message is checked to ensure that the IP is correct)->No data Breach
Checking of the proper user rights of the respective subsystems
When creating and using the algorithms, care is taken to ensure that no private information is used as output parameters (patient information, etc.). The output parameters (type, length, content) are fixed.
All accesses and manipulations to the system are logged
All connections where data is exchanged are logged.
All changes to the AI models are logged
There are administrative interfaces with which all accesses and manipulations to the system can be tracked.
There are administrative interfaces with which all connections can be tracked.
There are administrative interfaces that can be used to track all changes to the AI models.
The data structures of the stored data are created in such a way that individual entities can be deleted at any time without affecting other entities. Entities can also be deleted automatically after a time to be configured.

A further aspect of the present disclosure is a local (secure) data system that may, e.g., be provided for securing data. It may comprise means for receiving computer code from a patient assessment system, e.g., from an AI based patient assessment system as outlined herein. The computer code may be adapted to, when executed, process data to provide a training result for at least one AI based algorithm (of the patient assessment system), wherein the computer code is adapted to be executed in the local data system to process data stored in the local data system, e.g., as outlined herein. The local data system may further comprise means for executing the computer code to generate the training result. Further, the local data system may comprise means for providing the training result to the patient assessment system. Hence, the data may effectively be used by the patient assessment system for training without the data leaving the local data system.

The means (of the local data system) for receiving the computer code may be adapted to receive the computer code in a secured manner from the patient assessment system. Additionally or alternatively, the means for providing the training result may be adapted to provide the training result to the patient assessment system in a secured manner. Also here, a secure connection (e.g., the same secure connection) and/or a Blockchain (e.g., the same Blockchain) and/or suitable encryption schemes may be used. For example, the local data system and the patient assessment system may use a single secure connection and/or a single Blockchain.

The local data system may be a data system of a hospital, a health insurance company, a clinic network, a doctoral association, a medical IT-service provider, a health care provider, a network for medical services, a national network for health data, and/or an authority.

A further aspect of the present disclosure is a system comprising a patient assessment system for assessing a patient as described herein and a local data system as described herein.

A still further aspect of the present disclosure relates to an AI based method for assessing a patient. The method may be adapted to be carried out by a patient assessment system. The method may comprise the step of providing at least one AI based algorithm. The algorithm may be adapted to operate on data associated with the patient, e.g., as described herein.

The method may comprise the step of providing computer code to a local data system, wherein the computer code is adapted to, when executed, process data to provide a training result for the AI based algorithm, wherein the computer code is adapted to be executed in the local data system to process data stored in the local data system. The method may include providing the computer code to the local data system by a patient assessment system as described herein. The method may further include the step of receiving the training result from the local data system upon execution of the computer code in the local data system. The method may include receiving the training result from the local data system by a patient assessment system as described herein. The method may comprise one or more, or all steps described herein with respect to functions of the patient assessment system.

Yet another aspect of the present disclosure relates to a method that may be implemented by a local data system. The method may be implemented for securing data. The method may comprise the step of receiving computer code from a patient assessment system, e.g., as described herein. The computer code may be adapted to, when executed, process data to provide a training result for at least one AI based algorithm (e.g., of the patient assessment system). The computer code may be adapted to be executed in a local data system to process data stored in the local data system. The method may further include receiving the computer code from a patient assessment system by a local data system. The method may further comprise the step of executing, e.g., by the local data system, the computer code to generate the training result. Further, the method may include the step of providing the training result to the patient assessment system, e.g., by the local data system. The method may further comprise one or more, or all steps described herein with respect to functions of the local data system.

Yet another aspect of the present disclosure relates to a method for assessing a patient while securing data. The method may include the step of providing computer code to a local data system, wherein the computer code is adapted to, when executed, process data to provide a training result, wherein the computer code is adapted to be executed in the local patient system to process data stored in the local data system. The method may include providing the computer code to the local data system by a patient assessment system as described herein. The method may comprise the further step of receiving the computer code from the patient assessment system, e.g., by the local data system. The method may further comprise the step of executing, e.g., by the local data system, the computer code to generate the training result. Further, the method may include the step of providing the training result to the patient assessment system, e.g., by the local data system. The method may further include the step of receiving the training result from the local data system upon execution of the computer code in the local data system. The method may include receiving the training result from the local data system by a patient assessment system as described herein. The method may comprise one or more, or all steps described herein with respect to functions of the patient assessment system and/or the local data system.

A further aspect relates to a computer program. The computer program may comprise instructions which, when executed, cause a computer (and/or the means generally described herein) to perform the steps according to any of the methods as described herein.

The aspects described herein with respect to a patient assessment system may generally also be applied to any medical decision system that assists patients and/or medical staff. For example, the system may be implemented as a virtual coach providing assessment in the form of feedback and/or suggestions to a patient and/or medical staff. Additionally or alternatively, it may also be implemented as a system that provides assessment in the form of desired settings of a patient-specific medical devices (e.g., directly to the device, the patient, and/or medical staff).

Whether described as method steps, computer program and/or means, the functions described herein may be implemented in hardware, software, firmware, and/or combinations thereof. If implemented in software/firmware, the functions may be stored on or transmitted as one or more instructions or code on a computer-readable medium.

Computer-readable media include both computer storage media and communication media including any medium that facilitates transfer of a computer program from one place to another. A storage medium may be any available media that can be accessed by a general purpose or special purpose computer. By way of example, and not limitation, such computer-readable storage media can comprise RAM, ROM, EEPROM, FPGA, CD/DVD or other optical disk storage, magnetic disk storage or other magnetic storage devices, or any other medium that can be used to carry or store desired program code means in the form of instructions or data structures and that can be accessed by a general-purpose or special-purpose computer, or a general-purpose or special-purpose processor.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

FIG. 1 shows an example of a system including an exemplary patient assessment system and exemplary local data systems according to the present disclosure.

### DETAILED DESCRIPTION

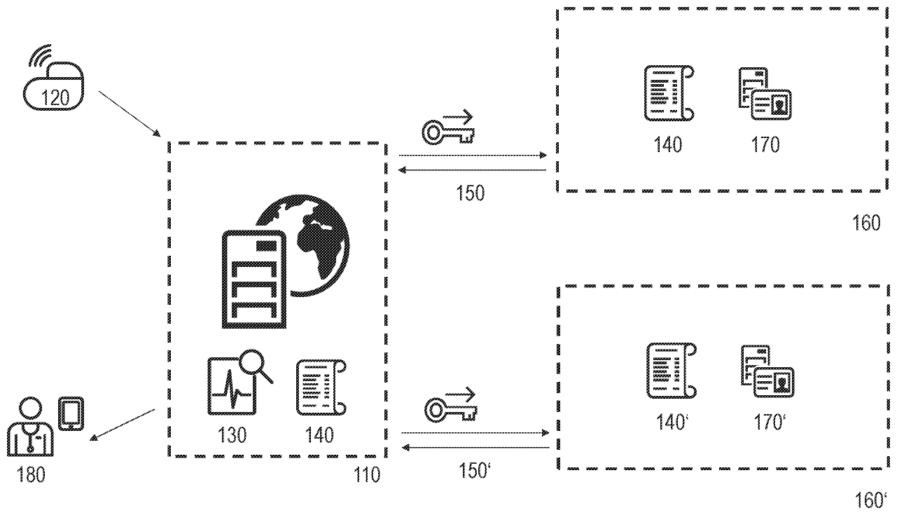

FIG. 1 shows a possible example for a patient assessment system 110. It may be realized as a (medical) decision system. It may be implemented as a central, server- and/or cloud-based system. It may be globally accessible. In an example, patient assessment system 110 may be implemented with the Home Monitoring Service Center (HMSC) of the applicant.

The patient assessment system 110 may comprise one or more embedded applications implementing the functions described herein. The patient assessment system 110 may be accessible from all over the world, e.g., via the internet. For example, one or more medical devices 120, such as medical implants, implanted or otherwise associated with a patient, may transmit data to the patient assessment system 110. In some examples, an intermediate unit (not shown) may receive data (locally, wirelessly, e.g., via WiFi, Bluetooth, 3G, 4G or 5G, etc.) transmitted by the one or more medical devices 120, and the intermediate unit may then transmit the data to the patient assessment system 110, e.g., over the internet. The patient assessment system 110 may thus receive data, in particular medical-device-specific data. The data may be associated with a patient, into whom the medical-device is implanted and/or who carries, wears, and/or is otherwise connected to the medical device. A secure connection may be used for transmission, and the data may be encrypted. Similarly, the patient assessment system 110 may transmit data to medical staff and/or the patient 180. Specifically the data may be transmitted to the intermediate unit at the patient and/or to a processing device accessible by the medical staff and/or to a smartphone of the patient (e.g., via the internet and/or using any of the communication technologies described herein).

The data received by the patient assessment system 110 from the medical devices 120 may generally only include data that can be acquired by the medical devices 120 themselves and may not include further patient data, such as current information on the patient's illness history or the patient's medication. The latter patient data may be sent to the patient assessment system 110 by the patient, or it may be acquired from other devices associated with the patient as described herein. Also, it may be acquired from a medical file of the patient, made available to the patient assessment system 110 and/or directly entered by the medical staff.

The patient assessment system 110 includes one or more algorithms 130 that may at least in part be AI based. Algorithm(s) 130 may be provided to (medically) assess a patient based on (medical) data associated with the patient. Algorithms 130 may be implemented by one or more embedded applications. Algorithms 130 may be adapted to assess a patient, e.g., provide a diagnosis, a recommended decision and/or a recommended action (to the patient and/or medical staff 180), based on medical-device-specific data and further patient data, e.g., in an automated manner.

The patient assessment system 110 may be adapted to provide computer code 140, 140′ to one or more local patient data systems 160, 160′. The computer code 140, 140′ may be implemented as one or more native applications (and/or smart applications and/or edge applications) that may be private. Patient assessment system 110 may comprise an (automatic) generator of such (native and/or training) applications corresponding to the computer code 140, 140′. The computer code 140, 140′ and/or the native application(s) may be generated (e.g., automatically) by patient assessment system 110 based on current data available for a patient. In other words, computer code 140, 140′ may be patient-specific and/or repeatedly generated based on the current data available concerning the patient. Also, computer code 140, 140′ may be generated (e.g., automatically) in a customized manner, adapted to the current issues to be resolved, e.g., as determined by the patient assessment system 110, e.g., as described herein, and/or adapted to local data system 160, 160′, as also described herein.

Patient assessment system 110 may comprise one or more interfaces to export computer code 140, 140′ to one or more local patient data systems 160, 160′. Corresponding computer code 140, 140′ may thus be distributed to any local data system 160, 160′ comprising useful training data. The computer code 140, 140′ may be specific to the local data system 160, 160′ and/or an access level, as described herein. An interface may generally refer to a hardware and/or a software interface. A hardware interface may be understood as, e.g., a USB connection, an ethernet connection or any other suitable hardware interface which allows a transfer of computer code 140, 140′. The hardware interface may be based on a wireless (e.g., Wi-Fi, 5G, LTE, Bluetooth or any other suitable standard and/or air interface) and/or a wired connection (e.g., a copper wired connection), e.g., via the internet. Additionally or alternatively, the interface may also be embedded in software which allows its export.

Computer code 140, 140′ may then be executed on local patient data system 160, 160′ and operate on the local (secure) patient data 170, 170′ within the bounds of the local patient data system 160, 160′. Hence, a multitude of patient data sets 170, 170′ may be made accessible for training. Only within local patient data system 160, 160′, access may be allowed to computer code 140, 140′ to access the local patient data 170, 170′ stored in local patient data system 160, 160′. Computer code 140, 140′ may be representative of a training task to be fulfilled based on the local patient data 170, 170′. Computer code 140, 140′ may be generated by patient assessment system 110, e.g., after algorithm 130 has operated on the data associated with the patient as available within the patient assessment system 110. Hence, computer code 140, 140′ may be specific to individual patients and/or to the specific question that currently needs to be answered for patient assessment. Consequently, also the answer to the question or, more generally, the result of the processing of the local patient data 170, 170′ by the computer code 140, 140′ in the local patient data system 160, 160′, that may then be returned to the patient assessment system 110 may be patient-specific and/or optimized based on currently available data for the patient.

Patient assessment system 110 may thus harness the (confidential) local patient data 170, 170′ for its training and its subsequent assessment in an optimum manner, but the local patient data 170, 170′ does not need to leave that local patient data system 160, 160′.

Computer code 140, 140′ may be transmitted to the local patient data system 160, 160′ via a secure connection 150, 150′ between the patient assessment system 110 and the respective local patient data system 160, 160′ (e.g., providing an encryption). The training result may then similarly be returned to the patient assessment system 110 via the secure connection 150, 150′ without patient data 170, 170′ being transmitted. The training result may be free from any aspects of the locally stored training data, such that from the training results, no conclusions can be drawn to patient-specific aspects stored in the training data. Additionally or alternatively, the provision of computer code 140, 140′ and/or the training result may be protected (e.g., from falsification, abuse, hampering, and/or ambiguity) using blockchain techniques and/or another suitable encryption schemes. For example, the computer code 140, 140′ and/or the training result may be stored in and retrieved from a blockchain.

The training result may then be used to update the algorithm(s) 130 in the patient assessment system. The training result may contain updated parameters for the algorithm(s). The updated algorithm(s) may then run on the patient assessment system 110 to provide an assessment of the patient, e.g., a diagnosis, a recommended decision and/or a recommended action. The assessment may then be transmitted to the patient and/or medical staff 180.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. An artificial intelligence based patient assessment system for assessing a patient, comprising:
at least one AI based algorithm;
means for providing computer code to a local data system wherein the computer code is adapted to, when executed, process data to provide a training result for the at least one AI based algorithm, wherein the computer code is adapted to be executed in the local data system to process data stored in the local data system;
means for receiving the training result from the local data system upon execution of the computer code in the local data system.

2. The system of claim 1, further comprising means for generating the computer code.

3. The system of claim 1, further comprising means for updating the at least one algorithm based on the training result.

4. The system of claim 3, wherein the updated algorithm is adapted for determining a medical condition of the patient at least in part based on data associated with the patient and based on the training result.

5. The system of claim 4, further comprising means for forwarding the determined medical condition to the patient and/or to medical staff.

6. The system of claim 2, wherein the algorithm and/or the updated algorithm are adapted to attempt determining a medical condition of the patient based on data associated with the patient; and the means for generating is adapted to generate, in case the medical condition of the patient cannot be determined based on the data associated with the patient, the computer code based on data associated with the patient, the computer code adapted for obtaining a training result required for determining the medical condition.

7. The system of claim 1, wherein the means for providing the computer code is adapted to provide the computer code to the local data system in a secured manner and/or the means for receiving the training result from the local data system is adapted to the receive the training result in a secured manner.

8. The system of claim 1, wherein the system is a cardiologic system.

9. A local data system comprising:
means for receiving computer code A from a patient assessment system, wherein the computer code is adapted to, when executed, process data to provide a training result for at least one artificial-intelligence based algorithm, wherein the computer code is adapted to be executed in the local data system to process data stored in the local data system;
means for executing the computer code to generate the training result;
means for providing the training result to the patient assessment system.

10. The system of claim 9, wherein the means for receiving the computer code is adapted to receive the computer code in a secured manner from the patient assessment system and/or the means for providing the training result is adapted to provide the training result to the patient assessment system in a secured manner.

11. The system of claim 1, wherein the local data system is at least one of a data system of: a hospital, a health insurance company, a clinic network, a doctoral association, a medical IT-service provider, a health care provider, a network for medical services, national network for health data, an authority.

12. System comprising a patient assessment system for assessing a patient according to claim 1.

13. An artificial-intelligence based method for assessing a patient comprising:
providing at least one AI based algorithm;
providing computer code to a local data system, wherein the computer code is adapted to, when executed, process data to provide a training result for the AI based algorithm, wherein the computer code is adapted to be executed in the local data system to process data stored in the local data system;
receiving the training result from the local data system upon execution of the computer code in the local data system.

14. A method, comprising:
receiving computer code from a patient assessment system, wherein the computer code is adapted to, when executed, process data to provide a training result for at least one artificial-intelligence based algorithm, wherein the computer code is adapted to be executed in a local data system to process data stored in the local data system;
executing the computer code to generate the training result;
providing the training result to the patient assessment system.

15. A computer program comprising instructions which, when executed, cause a computer to perform the steps according to the method of claim 13.
